(19) United States
(12) Patent Application Publication
(10) Pub. No.: US 2021/0260032 A1
(43) Pub. Date: Aug. 26, 2021

(54) COMPOSITION

(71) Applicant: Metaceutical Technologies Limited, London (GB)

(72) Inventor: Kenneth Benning, London (GB)

(21) Appl. No.: 17/261,548

(22) PCT Filed: Jul. 19, 2019
(86) PCT No.: PCT/GB2019/052020

§ 371 Date: Jan. 19, 2021

(30) Foreign Application Priority Data
Jul. 20, 2018 (GB) 1811889.3

(51) Int. Cl. A61K 31/407; A61K 36/71; A61K 31/235; A61K 47/02; A61K 47/34; C01B 39/02

(52) U.S. Cl. CPC A61K 31/407; A61K 36/71; C01B 39/02; A61K 47/02; A61K 47/34; A61K 31/235

(57) ABSTRACT

The invention relates to compositions comprising a chelating agent bound to an extended surface biologically inert substrate, methods of producing said compositions and uses of said compositions in treating heavy metal poisoning, as dietary supplements and as sports supplements.

The invention relates to compositions comprising a chelating agent bound to an extended surface biologically inert substrate, uses of said compositions and methods of producing said compositions.

Heavy metals within the body are both essential for health and deleterious, depending on the metals and their concentration. Entry to the body via diet and environment is not easily controlled and imbalance is increasingly thought to contribute to ill health. This invention describes a method of active control that can be continuously varied to manage varying conditions.

Symptoms that may arise from heavy metal poisoning include neurodevelopmental disorders such as intellectual disability in children, neurological disorders, central nervous system disorders, liver disease caused by hepatotoxicity, and kidney disease caused by nephrotoxicity. Deleterious heavy metals (including deleteriously high levels of essential heavy metals) can displace beneficial metals from their natural binding sites within cells leading to interruption of cellular homeostasis, and damage to nucleic acids, lipids, proteins and enzymes. Owing to their ability to interfere with and damage DNA and nuclear proteins, some heavy metals are known to be carcinogenic—for example, arsenic, chromium, cadmium, and nickel are classified by the International Agency for Research on Cancer as group 1 carcinogens. Deleterious heavy metals are also known to trigger metal-induced oxidative stress by enhancing the generation of reactive oxygen species (ROS, such as hydroxyl radical (HO.), superoxide radical (O₂.—) or hydrogen peroxide (H₂O₂)) in cells and/or by impairing cells' natural ability to detoxify ROS (e.g. through depletion of antioxidants and other free radical scavengers). Deleterious heavy metals have been linked to neurodegenerative diseases such as Alzheimer's disease and dementia, and oxidative stress has also been suggested to be a major intermediary risk factor in neurodegeneration associated with Alzheimer's disease (Huang et al. Biomed. Rep. 2016. 4(5):519-522).

Biological mechanisms exist to help remove heavy metals from the body. Heavy metals are excreted by three main routes: the urine, the bile and diffusion into the gut jejunum and ileum. These processes are largely driven by concentration gradients, although some, such as rubidium and copper, can be excreted against the gradient.

In the ileum, large quantities of fluid are secreted and later reabsorbed and will include some of the heavy metals excreted by the bile and jejunum. This phenomenon of “enterohepatic recirculation” can prevent efficient excretion of heavy metals via the digestive tract (J. P. K. Rooney, Toxicology, 2007. 234(3):145-156), as well as increased levels of heavy metals within the digestive tract (particularly in the jejenum and ileum).

Chelating agents have been used for the therapeutic reduction of heavy metals. Chelating agents are chemical compounds that can bind metal ions, and they have been employed to increase excretion of heavy metals from the body. The most widely used chelating agents include calcium-disodium EDTA, dimercaptosuccinic acid (DMSA) and dimercaptopropane sulfonate (DMPS), but these chelating agents are severely limited in their therapeutic applicability. For example, calcium-disodium EDTA and DMSA are approved by the Food and Drug Administration (FDA) for the chelation of lead, but administration of these chelating agents also leads to a loss of vitamin C and vitamin E and so supplementation is required to prevent detrimental side effects associated with deficiencies in these vitamins. Moreover, although DMSA increases excretion of arsenic, cadmium, lead and mercury, it also causes an undesirably high rate of copper loss. Similarly, DMPS chelates mercury, arsenic, lead and cadmium, but also leads to loss of beneficial elements such as copper, selenium, zinc and magnesium. While calcium-disodium EDTA, DMSA and DMPS are clinically useful in treating extreme cases of heavy metal poisoning, the requirement for careful monitoring for metal deficiencies and the need for essential metal and/or vitamin supplementation prevents the wide scale use of these agents in individuals with lower levels of heavy metal accumulation.

While chelating agents can lead to increased excretion of heavy metals, they can also mobilise heavy metals present in tissues (such as adipose tissue), leading to redistribution from tissues to vital organs, such as the brain (Sears, M. E. Scientific World Journal. 2013).

There exists an urgent and unmet need for further and improved compositions and methods for removing heavy metals from the body.

The need for further and improved compositions and methods for removing heavy metals from the body is provided by the present invention. The present invention also avoids many of the above-mentioned limitations associated with existing therapeutic chelating agents.

The present invention is based upon the surprising discovery that the activity of chelating agents may be focussed within the digestive tract by binding the chelating agent to an extended surface biologically inert substrate. Typically, the extended surface biologically inert substrate cannot pass through the gut-blood barrier, and so the activity of the chelating agent is retained and focussed within the digestive tract. Compositions of the invention are ideally-suited to oral administration, thereby avoiding the requirement for invasive administration methods such as injection. Advantageously, the compositions of the invention sequester heavy metals to the digestive tract for excretion, thereby reducing the level of “unbound” heavy metals which would otherwise enter the cycle of enterohepatic recirculation. Consequently, the compositions of the invention reduce the amount of heavy metals being circulated between the liver, bile, blood and digestive tract, and improve the efficiency of heavy metal excretion via the digestive tract. A further benefit is that compositions of the invention that are resident within the digestive tract can sequester heavy metals introduced to the body via the diet.

Put another way, the recycling of previously concentrated and excreted heavy metals can be reduced by the use of chelating agents or adsorbents in the lumen that will not release absorbed metals. Ideally, these materials are introduced by oral ingestion. Methods of reducing heavy metal levels within the body are known in the art, but they are limited by the lack of selectivity and removal of beneficial metals, sometimes to dangerously low levels. Consequently, invasive pathological tests are needed to measure metal deficiencies and prescribe metal supplements. These limitations are overcome by the present invention.

Compositions of the invention provide considerable advantages including throughout their passage through the digestive tract. Compositions of the invention are particularly efficacious when located at the jejunum and/or ileum because these are the main regions of the digestive tract that contribute to enterohepatic recirculation.

The inventors have identified melanin as an advantageous chelating agent for use in compositions of the invention. Melanin is a non-toxic metal chelating agent that is widely available and at low cost. However, ingested melanin can be absorbed through the gut-blood barrier, and so large doses of (un-bound) melanin would be required to ensure that a sufficient amount of the ingested melanin, with its heavy metal burden, is excreted. By binding melanin to an extended surface biologically inert substrate which does not pass through the gut-blood barrier, compositions of the invention advantageously avoid the high dosage requirements that would otherwise be required to achieve useful levels of metal chelation using melanin.

The inventors have identified zeolite as an advantageous extended surface biologically inert substrate for use in compositions of the invention. Zeolites are microporous minerals that are also widely available at low cost. Zeolite powders are substantially unable to pass through the gut-blood barrier and so provide an advantageous carrier for chelating agents e.g. melanin.

A further advantage of using zeolite as the extended surface biologically inert substrate is that it also possesses metal chelation properties. The inventors believe that compositions of the invention which comprise melanin as the chelating agent and zeolite as the extended surface biologically inert substrate achieve a synergistic heavy metal chelation activity.

Thus, in one embodiment, the gut-absorption disadvantages of unbound melanin are overcome by adsorbing the melanin, in a very thin layer, onto an extended surface biologically inert substrate such as a 13x zeolite.

Compositions of the invention are useful not only for the treatment of heavy metal poisoning, but also for use as a dietary supplement. When consumed as a dietary supplement, particularly when taken over a period of time, the composition of the invention advantageously suppresses the level of heavy metals in the subject. By suppressing heavy metals levels over time, the composition of the invention can advantageously reduce the risk of developing (helping prevent) disorders associated with heavy metals. As noted above, disorders associated with heavy metals include neurodegenerative disorders such as Alzheimer's disease and dementia. By reducing levels of heavy metals, compositions of the invention may also be used to treat or prevent neurodegenerative disorders. Moreover, by suppressing heavy metal levels over time, the composition of the invention can advantageously reduce the risk of developing (helping prevent) disorders associated with elevated levels of ROS, such as cancer. By reducing levels of heavy metals, compositions of the invention may also be used to treat or prevent cancer. Thus, compositions of the invention are useful for the treatment and prevention of disorders associated with heavy metals.

Compositions of the invention also provide considerable advantages when consumed as a dietary supplement before, during and/or after sport (i.e. as a sports supplement). Adipose tissue acts as a storage site for heavy metals in the body. The inventors believe that exercise (and concomitant reduction of adipose tissue) leads to release of heavy metals from adipose tissue to the rest of the body which can ultimately enter the digestive tract. When consumed as a dietary supplement before, during and/or after sport, the composition of the invention advantageously suppresses spikes in heavy metal concentrations that can accompany exercise. Moreover, it is well-known that exercise can lead to elevated ROS levels within the body—the improved ROS metabolism achieved by administration of the composition of the invention helps the subject detoxify ROS thereby further reducing the risk of developing disorders associated with elevated levels of ROS, such as cancer.

Having identified melanin and zeolite as highly desirable moieties for use in compositions of the invention, the inventors encountered challenges when attempting to bind these moieties together. These challenges have been overcome by methods of the invention.

Thus, the compositions of the invention provide several key benefits:
(1) accumulated heavy metals that are mobilised to the liver and excreted to the digestive tract in bile are bound by the composition of the invention, thereby reducing enterohepatic recirculation;
(2) heavy metals ingested from food or drink are bound by the composition of the invention absorption from the digestive tract; and
(3) the composition of the invention does not contribute to redistribution of heavy metals within the body because bound heavy metals are prevented from being absorbed from the digestive tract.

The compositions of the invention are useful in the treatment or prevention of any disease or symptom caused by, or exacerbated by, an accumulation of heavy metals.

The invention provides a composition comprising a chelating agent bound to an extended surface biologically inert substrate. In one embodiment, the chelating agent is a heavy metal chelating agent.

In one embodiment, the chelating agent is melanin. In one embodiment, the melanin is natural melanin. In one embodiment, the natural melanin is a Nigella sativa L. melanin extract.

In one embodiment, the chelating agent is tannin. In one embodiment, the tannin is natural tannin. In one embodiment, the natural tannin is an extract from a nut, such as hazelnut, walnut or almond.

In one embodiment, the extended surface biologically inert substrate is a zeolite. In one embodiment, the zeolite is 13X zeolite. In one embodiment, the zeolite is clinoptinolite. In one embodiment, the clinoptinolite is crushed natural clinoptinolite. In one embodiment, the clinoptinolite is synthetic clinoptinolite.

In one embodiment, the chelating agent is bound directly to the extended surface biologically inert substrate. In one embodiment, the chelating agent is integrated within the structure of the extended surface biologically inert substrate.

In one embodiment, the composition of the invention further comprises an adherent. In one embodiment, the adherent is Nafion. In one embodiment, binding of the chelating agent to the extended surface biologically inert substrate is enhanced by the adherent.

In one embodiment, the composition is in dosage form.

In one embodiment, the composition is formulated for oral administration. In one embodiment, the composition is in the form of a capsule. In one embodiment, the composition is in the form of a tablet. In one embodiment, the composition is in the form of a powder.

In one embodiment, the composition further comprises a gastro-resistant coating. In one embodiment, the composition is a time-release formulation. In one embodiment, the composition is a time-release formulation in the form of a capsule. In one embodiment, the composition further comprises an antacid.

In one embodiment, the composition is for use as a dietary supplement in a subject.

The invention also provides a dietary supplement comprising the composition of the invention.

In one embodiment, the composition of the invention is for use in treating heavy metal poisoning in a subject. In one embodiment, said treating further comprises administering a further chelating agent.

In one embodiment, the composition of the invention is for use in treating or preventing a neurodegenerative disorder in a subject.

In one embodiment, the composition of the invention is for use in treating or preventing cancer.

The invention also provides a method of treating heavy metal poisoning in a subject, the method comprising administering to the subject a composition of the invention. In one embodiment, the method further comprises administering to the subject a further chelating agent.

The invention also provides a method of treating or preventing a neurodegenerative disorder in a subject, the method comprising administering to the subject a composition of the invention.

The invention also provides a method of treating or preventing cancer in a subject, the method comprising administering to the subject a composition of the invention.

In one embodiment, the neurodegenerative disorder is selected from Alzheimer's disease and dementia.

In one embodiment, the subject is a human. In one embodiment, the subject is an animal, optionally wherein the subject is cow, sheep, pig, poultry, cat or dog.

In one embodiment, prior to administration of the composition to the subject, the level of heavy metal(s) are measured in the subject. In one embodiment, excess level of heavy metal(s) are identified in the subject. In one embodiment, the level of heavy metal(s) in the subject are assessed subsequent to administration of the composition to the subject. In one embodiment, upon identification of excess level of heavy metal(s) in the subject, said composition is re-administered to the subject. In one embodiment, upon identification of a deficiency in one or more beneficial heavy metal(s) in the subject, corresponding heavy metal supplement is/are administered to the subject.

In one embodiment, the level of heavy metal(s) in the subject are measured using a spectrometer. In one embodiment, the spectrometer is a miniature spectrometer.

The invention also provides a method for producing the composition of the invention, the method comprising: a) mixing the chelating agent with the extended surface biologically inert substrate; and b) agitating the mixture.

In one embodiment, agitating the mixture comprises subjecting the mixture to cavitation. In one embodiment, agitating the mixture comprises subjecting the mixture to sonication. In one embodiment, the sonication is ultrasonication. In one embodiment, the ultrasonication is applied by an ultrasonic horn. In one embodiment, the ultrasonic horn is a Barbell Ultrasonic Horn.

The invention also provides a method for producing the composition of the invention, the method comprising synthesising the extended surface biologically inert substrate in the presence of the chelating agent. In one embodiment, the extended surface biologically inert substrate is clinoptinolite. In one embodiment, the chelating agent is melanin.

In one embodiment, the method comprises: a) mixing melanin with an alkaline source, a silica source, and an alumina source; and b) applying hydrothermal conditions to the mixture.

Chelating agents are compounds that react with metal ions to form a stable complex. Chelating agents may also be referred to as chelants, chelators, chelation agents, or sequestering agents. In one embodiment, the chelating agent is a naturally-occuring chelating agent, such as a biomolecule, e.g. a pigment, protein, polysaccharide, polynucleic acid or amino acid (e.g. cysteine, alanine, histidine and glutamic acid). In one embodiment, the chelating agent is a synthetic chelating agent, e.g. DMSA, DMPS, ethylenediaminetetraacetic acid (EDTA), pentetic acid (DTPA), or ethylenediamine-N,N′-bis(2-hydroxyphenylacetic acid) (EDDHA).

In one embodiment, the chelating agent of the invention is melanin. Melanin is a pigment known to exhibit strong antioxidant activity. Melanin is reported to bind to a range of heavy metals and ROS. Melanin comprises oligomers and polymers of 5,6-dihydroxyindole (DIH) and 5,6-dihydroxyindole-2-carboxylic acid (DHICA) monomers. Metal binding is achieved by the amine and catechol hydroxyl groups of DHI and DHICA, and the carboxylic group of DHICA (Mauro, E. D. et al. MRS Communications 2017. 7(2):141-151). Melanin is well-suited for use in compositions of the invention because it binds more strongly to heavy metals than to other metals (Hong L. and Simon J., J Phys Chem B. 2007. 111(28):7938-7947). Thus, melanin binds preferentially to metals that are typically associated with heavy metal poisoning. A further therapeutic advantage provided by melanin's binding preference for heavy metals is that beneficial metals that might have been sequestered by melanin would typically be displaced by heavy metals subsequently encountered within the subject.

In one embodiment, the chelating agent is natural melanin. In one embodiment, the melanin is an extract from Nigella sativa L. Melanin extracted from Nigella sativa L. is commercially advantageous due to its natural abundance, high availability and low cost. In one embodiment, the melanin is an extract from Echinacea. In one embodiment, the melanin is synthetically-produced melanin.

This invention can also use other chelating agents such as tannins. Thus, in one embodiment, the chelating agent of the invention is tannin. Tannins are polyphenolic compounds known to bind various heavy metals including iron and mercury (Karamac, M. Int. J Mol. Sci. 2009. 10(12):5485-5497; and Torres, J. et al. J. Radioanal. Nucl. Chem. 1999. 240(1):361-365) and are known to exhibit antioxidant activity (Teissedre P. L. J. Sci. Food. Agric. 1996. 70:55-61). In one embodiment, the tannin is a naturally-occurring tannin. In one embodiment, the tannin is an extract from a nut. In one embodiment, the tannin is an extract from hazelnut, walnut or almond.

In one embodiment, the composition comprises more than one chelating agent. In one embodiment, the composition comprises melanin and tannin.

Chelating agents (alone or with their heavy metal burden) are typically absorbed from the gut preventing efficient excretion via the digestive tract. Advantageously, the inventors have determined that binding chelating agents to an extended surface biologically inert substrate leads to retention of the chelating agents (alone or with their heavy metal burden) within the digestive tract, enabling excretion via the faeces. Typically, an extended surface biologically inert substrate of the invention does not pass through the gut-blood barrier, either when bound to the chelating agent or when the extended surface biologically inert substrate is unbound.

The skilled person will appreciate that advantages of the invention are also achieved when the extended surface biologically inert substrate can pass through the gut-blood barrier in its unbound form, but not when bound to chelating agents.

Methods of determining whether a compound can pass through the gut-blood barrier are known in the art. Said methods include in vitro intestinal models, which measure the permeability of gut tissue to the compositions of the invention. One such in vitro model is an “Ussing chamber” which is a widely used physiological system to measure the transport of ions, nutrients, and drugs across various epithelial tissues. A guide to using Ussing chambers is provided by Clark L. American Journal of Physiology. 2009. 296(6):G1151-G1166. In vivo methods may also be employed involving comparing the amount of conjugate administered to a subject with the amount of compound excreted by the subject. In some embodiments, the model involves use of labelled conjugate to aid detection e.g. a radiolabel or a fluorescent label.

Typically, the extended surface biologically inert substrate of the invention comprises pores that increase the surface area of the substrate. In one embodiment, the extended surface biologically inert substrate of the invention is a microporous material.

Typically, the extended surface biologically inert substrate of the invention is insoluble and does not generate immune reactions in subjects following administration.

In one embodiment, the extended surface biologically inert substrate of the invention is zeolite. Zeolite is a microporous, aluminosilicate that has metal chelating properties. In one embodiment, the zeolite is the sodium form of zeolite X (13x zeolite). Zeolites of the invention include, but are not limited to, Linde Type A, Linde Type X, Linde Type L, offretite, erionite, RHO, PAU and KFI. In one embodiment, the zeolite is a natural zeolite. In one embodiment, the zeolite is a synthetic zeolite.

In one embodiment, the zeolite is clinoptinolite. In one embodiment, the clinoptinolite is crushed, natural clinoptinolite. In one embodiment, the clinoptinolite is synthetically-produced clinoptinolite.

In one embodiment, the zeolite is 13x zeolite. In one embodiment, the 13x zeolite is crushed, natural 13x zeolite. In one embodiment, the 13x zeolite is synthetically-produced 13x zeolite.

Thus, in one embodiment, the composition comprises melanin bound to zeolite. In one embodiment, the composition comprises melanin bound to clinoptinolite. In one embodiment, the composition comprises melanin bound to 13x zeolite.

Zeolites are particularly advantageous for use in compositions of the invention. Zeolites have ion exchange properties and are known to chelate heavy metals when in close proximity. Thus zeolite further increases heavy metal excretion by: 1) preventing absorption of the chelating agent and bound heavy metals through the gut-blood barrier; and 2) increasing the heavy metal binding capacity of the composition.

In one embodiment, chelating agent is bound to the surface of the extended surface biologically inert substrate.

In one embodiment, chelating agent is integrated within the structure of the extended surface biologically inert substrate.

In one embodiment, chelating agent is bound indirectly to the extended surface biologically inert substrate via an adherent. An adherent may be used to further improve the binding of the chelating agent to the extended surface biologically inert substrate. In one embodiment, the composition comprises an adherent. In one embodiment, the adherent is a sulfonated tetraflurorethylene, e.g. Nafion. In one embodiment, the extended surface biologically inert substrate is pre-coated with an intermediate layer, for example Nafion, to improve adherence of the chelating agent.

In one embodiment, the weight ratio of chelating agent to extended surface biologically inert substrate in the composition is 1:1. In one embodiment, the weight ratio of chelating agent to extended surface biologically inert substrate in the composition is at least 2:1. In one embodiment, the weight ratio of chelating agent to extended surface biologically inert substrate in the composition is at least 4:1. In one embodiment, the weight ratio of chelating agent to extended surface biologically inert substrate in the composition is at least 8:1.

In one embodiment, the weight ratio of extended surface biologically inert substrate to chelating agent in the composition is at least 2:1. In one embodiment, the weight ratio of extended surface biologically inert substrate to chelating agent in the composition is at least 4:1. In one embodiment, the weight ratio of extended surface biologically inert substrate to chelating agent in the composition is at least 8:1.

In one embodiment, compositions of the invention comprise particles of chelating agent bound to extended surface biologically inert substrate. The skilled person will appreciate that the particle size will depend upon the relative sizes of the chelating agent and the extended surface biologically inert substrate. The skilled person will also be aware that distribution of particle sizes may be polydisperse. The method of binding chelating agent to extended surface biologically inert substrate may also affect particle size. In one embodiment wherein the (i) chelating agent is melanin, (ii) the extended surface biologically inert substrate is clinoptinolite, and (iii) particles of the invention were formed using ultrasonication, at least 30% (e.g. at least 40%, at least 50%, at least 60% or at least 70%) of the particles are 20-200 μm in diameter. Said percentage values correspond to weight percentages in the polydisperse particle mixture, and said diameter corresponds to the diameter at the widest part of the particles.

The compositions of the invention are ideally suited to oral administration. Oral administration is the preferred route of administration because it does not require the involvement of a skilled practitioner, and it avoids the discomfort associated with intravenous and intramuscular administration required by most existing chelation therapies.

In one embodiment, the composition of the invention is formulated in dosage form.

In one embodiment, the composition of the invention is formulated for oral administration. In one embodiment, the composition is formulated for oral administration with sweeteners or flavouring agents.

The composition of the invention (e.g. coated zeolite) can be in the form of powder or small balls, compressed to a tablet or contained in a capsule. In one embodiment, the composition is in a powder form. In one embodiment, the composition is in a tablet form. In one embodiment, the composition is in a capsule form.

This enables a much more efficient and precise use of melanin that is then confined within the gut and that will be eliminated in the faeces. A further advantage is that the tablets or capsules of the invention can be given a protective coating, such as a gastro-resistant coating to enable positional or slow release as desired. For example, adsorbing the metals concentrated in the bile at the top of the gut, or being activated only when the ileum is reached. This also enables a degree of targeting specific metals. Another advantage is that applying the chelating agent in a thin coat on an extended surface gives more effective use of the agent and a more precise measurement of the adsorptive capacity, enabling dosing to be more accurate.

Thus, in one embodiment, compositions of the invention comprise a gastro-resistant coating. Gastro-resistant coatings reduce or prevent the dissolution and disintegration of the composition in the gastric environment. The gastro-resistant coating also prevents potentially beneficial metals present in food from being sequestered by the composition of the invention while the composition is in the stomach. Gastro-resistant coatings can also be used to help target the composition of the invention to the intestine (after it has transited the stomach). Advantageously, this helps increase the concentration of composition of the invention within the intestine.

Gastro-resistant coatings are typically selected from fatty acids, waxes, shellac, plastics and plant fibers, and include e.g. hydroxypropyl methyl cellulose phthalate, methyl acrylate-methacrylic acid copolymers, cellulose acetate succinate, cellulose acetate trimellitate, sodium alginate, hydroxypropyl methyl cellulose acetate succinate (hypromellose acetate succinate), polyvinyl acetate phthalate (PVAP), cellulose acetate phthalate (CAP), zein, methyl methacrylate-methacrylic acid copolymers and enteric coating aqueous solution (ethylcellulose, medium chain triglycerides, oleic acid, sodium alginate, stearic acid).

In one embodiment, the composition is a time-release formulation. Time-release formulations include sustained release formulations (where prolonged release is intended), pulse release formulations and delayed release formulations (e.g. to target different regions of the digestive tract). Time-release formulations of the invention typically allow the composition to be released gradually into the digestive tract (e.g. to distribute the formulation throughout the digestive tract) or to be released in a delayed manner (e.g. to delayed release of the composition until the small intestine has been reached). Time-release formulations are known in the art, and typically include e.g. polymeric based components or coating membranes. Advantageously, the use of time-release formulation helps increase the concentration of composition of the invention within the intestine. The release time for the time-release formulations of the invention may be dependent on the formulation and may range from minutes to hours to days. Typically, time-release formulations of the invention are timed to release within 3 to 10 hours, optionally 4 to 8 hours, optionally 6 to 8 hours following ingestion.

In one embodiment, time-release formulations of the invention are formulated for delivery to the small intestine. In one embodiment, time-release formulations of the invention are formulated for delivery to the jejunum and ileum

In one embodiment, the composition of the invention comprises a gastro-resistant time release coating.

Compositions of the invention may further comprise an excipient, such as a pharmaceutically acceptable excipient. The excipient may comprise one or more of a preservative, a buffer, an antacid, a bulking agent, filler, flavour, colour or sweetener.

The use of gastro-resistant and/or time-release formulations is advantageous because they can help concentrate the composition of the invention at the intended location within the digestive tract. Moreover, the use of gastro-resistant and/or time-release formulations can help prevent the composition of the invention from sequestering metals before the composition reaches the intended location within the digestive tract. Advantageously, this helps ensure that the composition of the invention retains maximum potency at the intended location within the digestive tract.

The use of gastro-resistant and/or time-release formulations is particularly advantageous when the chelating agent is melanin. The chelating activity of melanin is known to be dependent upon its degree of hydration—at low hydration (e.g. less than 5% hydrated), melanin exhibits relatively low chelation activity, and its chelation activity increases with the degree of hydration. The use of gastro-resistant and/or time-release formulations can help prevent exposure of melanin to moisture until it reaches the intended location within the digestive tract. By exploiting the hydration-dependent chelation properties of melanin, the present invention further ensures that the composition of the invention retains maximum potency at the intended location within the digestive tract.

This technique gives control over the amount and position of heavy metal removal and to some degree the type, because composition and concentration vary along the length of the gut. Knowledge of the quantity and type of metals removed enables accurate metal supplements to be calculated.

Compositions of the invention are ideally suited to use in chelation therapy. The invention provides a composition comprising a chelating agent bound to an extended surface biologically inert substrate for use in the treatment of heavy metal poisoning in a subject. Heavy metal poisoning (sometimes referred to as metal toxicity or metal poisoning) may occur when a subject is exposed to high levels of heavy metals in food, water, air and/or medicine. Subjects may also be exposed to high levels of heavy metals by industrial exposure.

Heavy metals are naturally occurring elements that have a high atomic weight. Heavy metals are used widely in industrial, agricultural and domestic applications and there are growing concerns over their potential effects on health and the environment. Heavy metals sometimes imitate the action of an essential element in the body, interfering with the metabolic process resulting in illness. Heavy metals typically accumulate in the body (and in the food chain) and so heavy metal poisoning is often chronic. Exposure to high levels of heavy metals can also lead to acute heavy metal poisoning.

Metals such as calcium, zinc, iron, chromium, copper, cobalt, nickel, magnesium, sodium and potassium are essential for health, but they are deleterious to health when present at elevated concentrations. For some metals such as chromium and copper, there is a very narrow range of concentration between the provision of beneficial and toxic effects. Other metals, however, have no established biological function and are generally considered to be non-essential metals—these include metals such as aluminium, antinomy, arsenic, barium, beryllium, bismuth, cadmium, gallium, germanium, gold, indium, lead, lithium, mercury, nickel, platinum, silver, strontium, tellurium, thallium, tin, titanium, vanadium and uranium (Tchounwou P. et al. Exp Suppl. 2012. 101:133-164). Certain metals such as arsenic, cadmium, chromium, lead, manganese, mercury and the radioactive metals are highly toxic.

The invention also provides a method of treating heavy metal poisoning in a subject, the method comprising administering a composition of the invention to said subject.

The dosage regimen will be determined, at least in part, by the need of the individual and be dependent upon the judgment of the practitioner (e.g. doctor or veterinarian). For therapeutic applications, the dose is typically a therapeutically effective amount of composition of the invention. Those of ordinary skill in the art will appreciate that, in some embodiments, a therapeutically effective amount is formulated and/or administered in a single dose. In some embodiments, a therapeutically effective agent is formulated and/or administered in a plurality of doses, for example, as part of a dosing regimen.

The compositions of the invention may be given in a single dose schedule (i.e. the full dose is given at substantially one time). Alternatively, the compositions of the invention may be given in a multiple dose schedule. A multiple dose schedule is one in which a primary course of treatment may be with 1-6 separate doses, followed by other doses given at subsequent time intervals required to maintain and/or reinforce the chelation activity, for example at 1-4 months for a second dose, and if needed, a subsequent dose(s) after a further 1-4 months.

The dose of composition administered to a subject with heavy metal poisoning can be altered depending on the concentration of heavy metals in the body.

In one embodiment, the composition of the invention is for use in the treatment of heavy metal poisoning in a subject, wherein the subject is further treated with an additional chelation therapy. Additional chelation therapies include therapies available in the art that can be orally, intravenously or intramuscularly delivered and include, but are not limited to, dimercaprol, DMSA, DMPS, penicillamine, calcium-di sodium EDTA, deferoxamine, deferasirox and deferiprone. Preferably, the additional chelation therapy increases excretion of heavy metals to the liver and subsequently into the ileum in bile. Use of the composition of the invention in combination with a liver excretion biased additional chelation therapy can provide synergistic effects for the treatment of heavy metal poisoning. In more detail, the additional chelating agent increases mobilisation of heavy metals to the liver, from which they are excreted in bile into the ileum. The composition of the invention binds heavy metals excreted in the bile and prevents their reabsorption from the digestive tract, thereby increasing the efficiency of heavy metal excretion.

In one embodiment, the composition of the invention and the additional chelating agent are administered to a subject simultaneously. Simultaneous administration means administration at (substantially) the same time.

In one embodiment, the additional chelating agent and composition of the invention are administered to a subject sequentially. Administration may be separated by hours, e.g. the chelating agent is administered and then the composition of the invention is administered after 1 hour, after 2 hours, after 3 hours, after 4 hours, after 5 hours, after 6 hours, after 12 hours, or after 24 hours, or vice versa. Administration may be separated by days, e.g. the chelating agent is administered and then the composition of the invention is administered after 1 day, after 2 days, after 3 days, after 4 days, after 5 days, after 6 days, or after 7 days, or vice versa.

The invention also provides a composition comprising a chelating agent bound to an extended surface biologically inert substrate for use as a dietary supplement in a subject.

The invention also provides a dietary supplement comprising a composition of the invention.

In one embodiment, the dietary supplement is a sports supplement.

In one embodiment, the composition of the invention is a dietary supplement for animal feed, such as domestic pet food or agricultural animal feed. Thus in one embodiment, the invention provides domestic pet food comprising composition of the invention (e.g. food for cats or dogs). In one embodiment, the invention provides agricultural animal feed comprising composition of the invention (e.g. food for cows, sheep, pigs or poultry).

A dietary supplement may also contain additional substances that are beneficial to nutrition or have a beneficial biological effect. In some embodiments, the composition for use as a dietary supplement comprises a second beneficial ingredient, e.g. a vitamin, a mineral, a herb, an amino acid, a supplement to increase total dietary intake, a flavourant, a colourant, a sweetener, or a concentrate, metabolite, constituent, extract, or combination of said additional ingredients.

Examples of flavourant for use in a dietary supplement include, but are not limited to, essential oils, oleoresin, essence, protein hydrolysate, distillate or any product of roasting, heating or enzymolysis of fruits or vegetables, anise extract, imitation banana extract, imitation cherry extract, chocolate extract, chocolate flavouring, lemon extract, orange extract, peppermint extract, imitation pineapple extract, imitation rum extract, imitation strawberry extract, or vanilla extract, volatile oils, e.g. balm oil, bay oil, bergamot oil, cedarwood oil, walnut oil, cherry oil, cinnamon oil, clove oil, peppermint oil. In one embodiment, the composition for use as a dietary supplement further comprises cocoa or chocolate.

The composition of the invention may further comprise emulsifiers. Emulsifiers improve stability of the final product. Suitable emulsifiers include, but are not limited to, lectin, monoglycerides and diglycerides. In addition to the carbohydrates described above, the nutritional supplement can contain natural or artificial sweeteners, e.g. (poly)saccharides.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola), dehydroepiandosterone (DHEA), green tea (polyphenols), inositol, kelp, dulse, bioflavonoids, maltodextrin nettles, niacin, niacinamide, rosemary, selenium, silica and spirulina. Such optional ingredients may be either naturally occurring or concentrated forms.

In some embodiments, the dietary supplement further comprises vitamins and minerals including, but not limited to, calcium phosphate, calcium acetate, potassium phosphate, magnesium sulfate, magnesium oxide, sodium chloride, potassium chloride, potassium acetate, ascorbic acid, ferric orthophosphate, riboflavin, beta-carotene, pyridoxine hydrochloride, thiamine mononitrate, folic acid, biotin, chromium chloride, potassium iodide, vitamin D, vitamin A, vitamin C, inositol, and potassium iodide. Suitable dosages for vitamins and minerals may be obtained, for example, by consulting the U.S. RDA guidelines.

In one embodiment, the dietary supplement is in the form of an energy bar, a meal replacement bar or a beverage. The dietary supplement generally comprises nutritional calories. Preferably, the dietary supplements provide carbohydrates, proteins, and fats. The dietary supplement may further comprise monosaccharides, disaccharides or polysaccharides, or a combination thereof. The dietary supplement may contain, combinations of sources of simple, medium and complex carbohydrates e.g. sucrose, maltodextrins, and uncooked corn starch.

In one embodiment, the dietary supplement is a sports supplement. Thus, the invention also provides a composition comprising a chelating agent bound to an extended surface biologically inert substrate for use as a sports supplement in a subject. The sports supplement may be in the form of a sports energy bar further comprising a source of carbohydrate, e.g. glucose. The sports supplement may be in the form of a beverage, e.g. an isotonic energy drink.

The composition for use as a sports supplement can relieve, reduce or prevent pain associated with exercise. In one embodiment, the composition according to the invention is for use in relieving or reducing muscular pain, and/or to relieve or reduce cramps. In one embodiment, the composition of the invention is for use in reducing the formation of lactic acid and/or for increasing the clearance of lactic acid. In one embodiment, the composition of the invention is for use in improving endurance during physical activity. Endurance may be defined as the capacity to maintain long-term, intense effort. The sports supplement of the invention may be taken before, during and/or after exercise.

In one embodiment, the sports supplement further comprises at least one active ingredient. Active ingredients include, but are not limited to, vitamin B, vitamin C, vitamin D, vitamin E, vitamin H, vitamin K, creatine, caffeine, nitrate, beta-alanine, glucosamine, guarana, glucomannan, capsaicinoids, capsaicin, inositol, sodium bicarbonate and protein hydrolysates.

The invention provides a method of reducing, relieving or preventing muscular pain or cramp, the method comprising administering a composition according to the invention to a subject before, during or after exercise.

In one embodiment, the composition of the invention is for use as a preventative measure when exposure to heavy metals is suspected in a subject.

In one embodiment, a subject according to the invention is a human. In one embodiment, the subject according to the invention is an animal, e.g. cow, sheep, pigs, poultry (e.g. chicken, turkey), cat or dog.

In one embodiment, a spectrometer is applied to the skin to estimate metal concentrations in a subject. In one embodiment, heavy metal concentrations are estimated in the subject using a miniature spectrometer. The concentration of heavy metals in the subject can be used to inform the dosage of the composition of the invention administered to the subject. In one embodiment, heavy metal concentrations measured in a subject are compared to healthy reference levels.

In one embodiment, when heavy metal concentrations are below the reference level, a lower dose of the composition may be administered to a subject, and/or the dosage interval may be increased. Wherein a heavy metal deficiency is identified by a reduced concentration of heavy metal in the subject compared to the healthy reference level, the method of the invention may comprise administering a beneficial metal supplement to the subject. Thus, in one embodiment, supplements comprising composition of the invention and beneficial heavy metals are administered to subjects identified as having heavy metal concentrations lower than healthy reference levels. In one embodiment, when heavy metal concentrations are higher than the healthy reference levels, the dose of the composition of the invention administered to the subject may be maintained or increased and/or the dosage interval may be decreased.

In one embodiment, the invention provides a composition comprising composition of the invention and one or more beneficial heavy metals, such as zinc and/or magnesium.

The addition of a telemetric link can be used to indicate the requirement for control or supplement to achieve optimal levels. This can easily extend to an automated pre-stocked pill dispenser.

The composition of the invention may be produced by any method that binds the chelating agent to the extended surface biologically inert substrate.

The inventors encountered challenges when attempting to bind melanin to zeolite. These challenges have been overcome by methods of the invention.

The invention provides a method of producing a composition comprising a chelating agent bound to an extended surface biologically inert substrate, the method comprising: a) mixing a chelating agent with an extended surface biologically inert substrate; and b) agitating the mixture.

In one embodiment, agitating the mixture comprises subjecting the mixture to cavitation. In one embodiment, agitation or cavitation is achieved by subjecting the mixture to sonication. In one embodiment, the sonication is ultrasonication. Ultrasonication involves applying frequencies of >20 kHz to the mixture. In one embodiment, the ultrasonication is applied by an ultrasonic horn. An ultrasonic horn is used to augment the oscillation displacement amplitude provided by an ultrasonic transducer. In one embodiment, the ultrasonic horn is a Barbell Ultrasonic Horn. In one embodiment, the sonication is Barbell Horn ultrasonication. Barbell Horn ultrasonication is desirable because it allows industrial scale application of ultrasonic frequencies without reducing ultrasonic amplitude or compromising product quality when compared to smaller scale production.

The invention also provides a method for producing a composition comprising a chelating agent bound to an extended surface biologically inert substrate, the method comprising synthesising the extended surface biologically inert substrate in the presence of the chelating agent. In one embodiment, the extended surface biologically inert substrate is clinoptinolite. In one embodiment, the chelating agent is melanin.

In one embodiment, the invention provides a method for producing a composition comprising melanin bound to clinoptinolite, the method comprising: a) mixing melanin with an alkaline source, a silica source, and an alumina source; and b) applying hydrothermal conditions to the mixture. In one embodiment, step b) of the method is performed at between 100 and 200° C.

Methods of producing clinoptinolite are known in the art (Ambrozova P. et al. Molecules. 2017. 22:1107). Methods of the invention include methods of producing clinoptinolite as disclosed in (Ambrozova P. et al. Molecules. 2017. 22:1107) wherein the reaction mixture further comprises melanin.

Briefly, clinoptinolite may be produced by mixing the raw materials for clinoptinolite (silica, alumina, and alkali) and exposing these materials to hydrothermal conditions, e.g. in an autoclave. In one embodiment, the composition of the invention is produced by adding a chelating agent (e.g. melanin) to the pre-autoclave formulation for clinoptinolite production (e.g. silica, alumina, and alkali). Silica may be provided to the pre-autoclave formulation in the form of colloidal silica, silica gel, fumed silica, and/or amorphous silica. Alumina may be provided to the pre-autoclave formulation in the form of aluminium hydroxide, sodium aluminate, aluminium, and/or aluminium salts. Alkali may be provided to the pre-autoclave formulation in the form of sodium hydroxide and/or potassium hydroxide.

A method of producing the composition of the invention may further comprise a step of determining the absorptive capacity of the composition. In one embodiment, the absorptive capacity is determined by a gravimetric method.

There now follows a brief description of the Figures, which illustrate aspects and/or embodiments of the present invention.

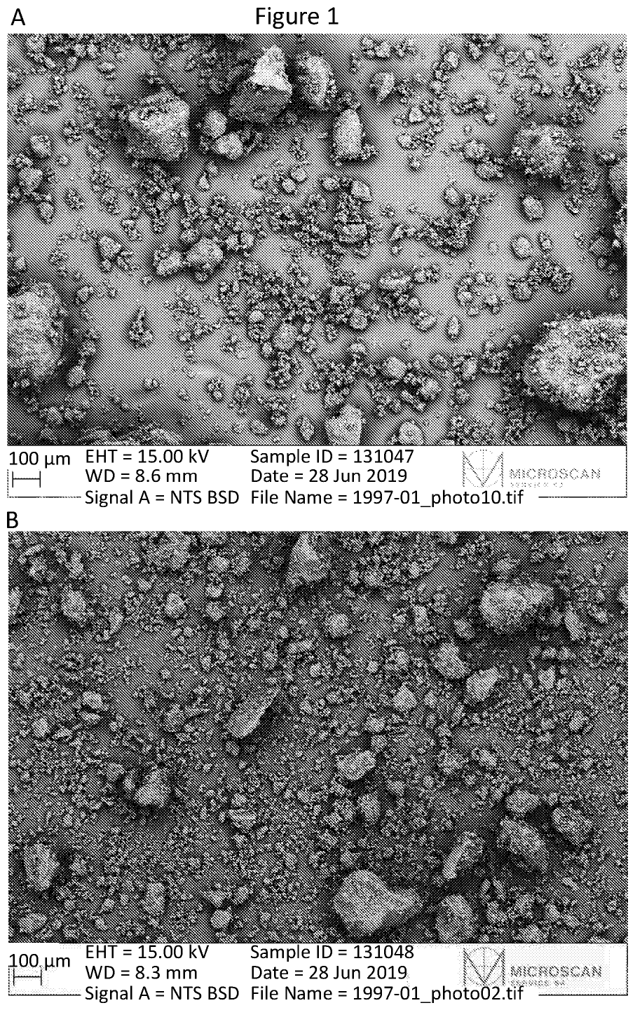

FIG. 1: Scanning electron microscopy image of (A) melanin and (B) zeolite.

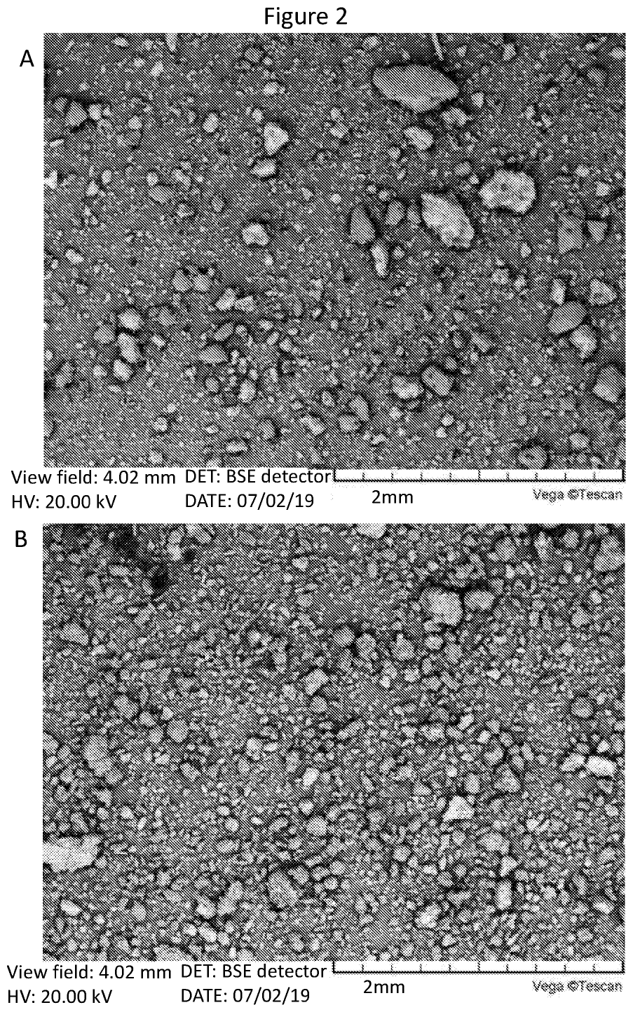

FIG. 2: Scanning electron microscopy image of zeolite and melanin (A) before ultrasonication and (B) after ultrasonication.

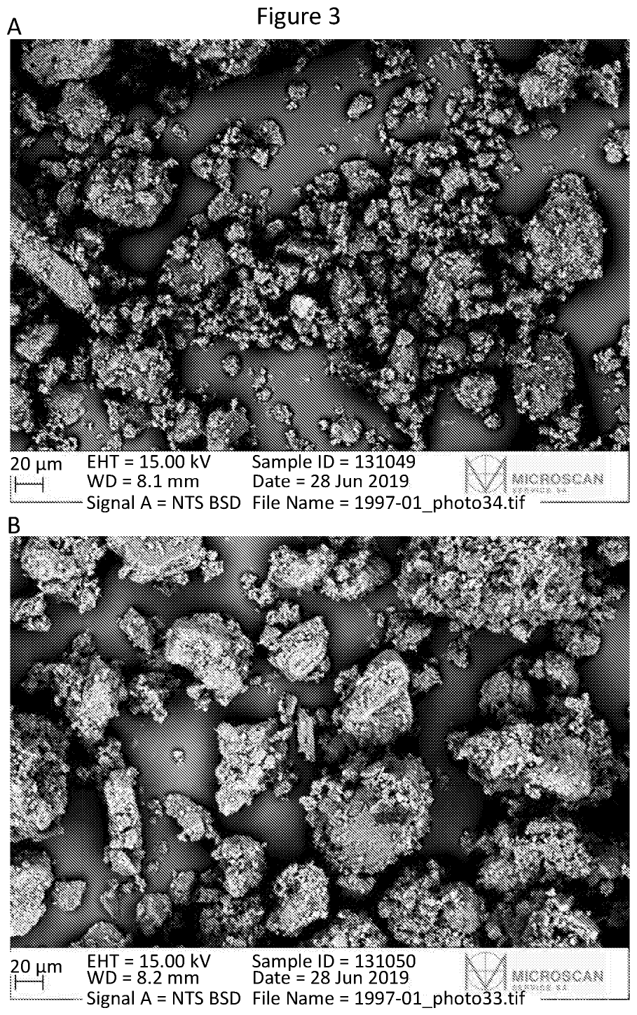

FIG. 3: Scanning electron microscopy image of zeolite and melanin (A) before ultrasonication and (B) after ultrasonication.

### EXAMPLES

### Example 1: Preparation of a Composition Comprising a Chelating Agent Bound to an Extended Surface Biologically Inert Substrate

The inventors studied melanin as the chelating agent and clinoptinolite as the extended surface biologically inert substrate. Scanning electron microscopy (SEM) images of melanin and zeolite powders used for the production of compositions of the invention are provided in FIG. 1A and 1B, respectively.

To obtain SEM images, samples were deposited on a specimen stub using electronically conductive double-sided adhesive tape and were sputter-coated with gold to avoid charging effect. Samples were visualised using field emission SEM to visualise the production of agglomerated particles of melanin and clinoptinolite.

Melanin and clinoptinolite powders were mixed, and a 1:1 weight ratio mixture of melanin and clinoptinolite is shown in FIGS. 2A and 3A.

The mixture of melanin and clinoptinolite was then agitated using Barbell Horn ultrasonication. Agitation of the powders led to binding of melanin to zeolite. FIGS. 2B and 3B demonstrate that, following agitation: (a) larger particles of melanin and clinoptinolite disintegrated; and (b) smaller particles of melanin and clinoptinolite agglomerated to form new, larger particles, which corresponded to melanin bound to clinoptinolite. The binding of melanin to clinoptinolite is clearly evident when comparing FIGS. 2A and 3A (pre-agitation mixtures) with FIGS. 2B and 3B (after agitation), which demonstrate a reduced distribution of particle size (increased uniformity of particle size) following agitation.

### Example 2 Preparation of a Composition Comprising a Chelating Agent Bound to an Extended Surface Biologically Inert Substrate by Zeolite Synthesis

The composition of the invention may be produced by synthesising zeolite (e.g. clinoptinolite) in the presence of melanin.

Clinoptinolite may be produced by methods described in Ambrozova P. et al. Molecules. 2017. 22:1107. The starting materials for clinoptinolite include sources of silica, alumina, and alkali. The proportion and source of starting materials can be varied leading to the different rates of formation of the clinoptinolites as described in Ambrozova P. et al. Molecules. 2017. 22:1107.

A method of producing the composition of the invention may involve mixing 2.1NaOH:1Al(OH)₃:5SiO₂:52.5H₂O and melanin. Alternatively, a method of producing the composition of the invention may involve mixing 2.1KOH:1Al(OH)₃:5SiO₂:52.5H₂O and melanin. These mixtures are exposed to a temperature in the range of between 100° C. and 200° C. for a period of time between 12 and 300 hours.

Silica may be provided to the mixture in the form of colloidal silica, silica gel, fumed silica, and/or amorphous silica. Alumina may be provided to the mixture in the form of aluminium hydroxide, sodium aluminate, aluminium, and/or aluminium salts. Alkali may be provided to the mixture in the form of sodium hydroxide and/or potassium hydroxide.

1) A composition comprising a chelating agent bound to an extended surface biologically inert substrate.

2) The composition according to claim 1, wherein the chelating agent is a heavy metal chelating agent.

3) The composition according to claim 1 or claim 2, wherein the chelating agent is melanin.

4) The composition according to claim 3, wherein the melanin is natural melanin.

5) The composition according to claim 4, wherein the natural melanin is a Nigella sativa L. melanin extract.

6) The composition according to claim 1 or claim 2, wherein the chelating agent is tannin.

7) The composition according to claim 6, wherein the tannin is natural tannin.

8) The composition according to claim 7, wherein the natural tannin is an extract from a nut, such as hazelnut, walnut or almond.

9) The composition according to any one of the preceding claims, wherein the extended surface biologically inert substrate is a zeolite.

10) The composition according to claim 9, wherein the zeolite is 13X zeolite.

11) The composition according to claim 9, wherein the zeolite is clinoptinolite.

12) The composition according to claim 11, wherein the clinoptinolite is crushed natural clinoptinolite.

13) The composition according to claim 11, wherein the clinoptinolite is synthetic clinoptinolite.

14) The composition according to any one of the preceding claims, wherein the chelating agent is bound directly to the extended surface biologically inert substrate.

15) The composition according to any one of the preceding claims, wherein the chelating agent is integrated within the structure of the extended surface biologically inert substrate.

16) The composition according to any one of the preceding claims, wherein the composition further comprises an adherent.

17) The composition according to claim 16, wherein the adherent is Nafion.

18) The composition according to claim 16 or claim 17, wherein binding of the chelating agent to the extended surface biologically inert substrate is enhanced by the adherent.

19) The composition according to any one of the preceding claims, wherein the composition is in dosage form.

20) The composition according to any one of the preceding claims, wherein the composition is formulated for oral administration.

21) The composition according to any one of the previous claims, wherein the composition is in the form of a capsule.

22) The composition according to any one of claims 1 to 20, wherein the composition is in the form of a tablet.

23) The composition according to any one of claims 1 to 20, wherein the composition is in the form of a powder.

24) The composition according to any one of claims 19 to 23, further comprising a gastro-resistant coating.

25) The composition according to any one of claims 19 to 24, wherein the composition is a time-release formulation.

26) The composition according to claim 25, wherein the composition is a time-release formulation in the form of a capsule.

27) The composition according to any one of claims 19 to 26, further comprising an antacid.

28) The composition according to any one of claims 1 to 27, for use as a dietary supplement in a subject.

29) A dietary supplement comprising the composition according to any one of the preceding claims.

30) The composition according to any one of the preceding claims for use in treating heavy metal poisoning in a subject.

31) The composition for use according to claim 30, wherein said treating further comprises administering a further chelating agent.

32) The composition according to any one of claims 1 to 29, for use in treating or preventing a neurodegenerative disorder in a subject.

33) The composition according to any one of claims 1 to 29, for use in treating or preventing cancer.

34) A method of treating heavy metal poisoning in a subject, the method comprising administering to the subject a composition according to any one of claims 1 to 29.

35) The method according to claim 34, the method further comprising administering to the subject a further chelating agent.

36) A method of treating or preventing a neurodegenerative disorder in a subject, the method comprising administering to the subject a composition according to any one of claims 1 to 29.

37) A method of treating or preventing cancer in a subject, the method comprising administering to the subject a composition according to any one of claims 1 to 29.

38) The composition for use according to claim 32 or the method according to claim 36, wherein the neurodegenerative disorder is selected from Alzheimer's disease and dementia.

39) The composition for use according to any one of claim 28, 30 to 33 or 38, or the method according to any one of claims 34 to 38, wherein the subject is a human.

40) The composition for use according to any one of claim 28, 30 to 33 or 38, or the method according to any one of claims 34 to 38, wherein the subject is an animal, optionally wherein the subject is cow, sheep, pig, poultry, cat or dog.

41) The composition for use according to any one of claims 28, 30 to 33 or 38 to 40, or the method according to any one of claims 34 to 40, wherein prior to administration of the composition to the subject, the level of heavy metal(s) are measured in the subject.

42) The composition for use or the method according to claim 41, wherein excess level of heavy metal(s) are identified in the subject.

43) The composition for use or the method according to any one of claims 28, 30 to 33 or 38 to 42, or the method according to any one of claims 34 to 42, wherein the level of heavy metal(s) in the subject are assessed subsequent to administration of the composition to the subject.

44) The composition for use or the method according to claim 43 wherein, upon identification of excess level of heavy metal(s) in the subject, said composition is re-administered to the subject.

45) The composition for use or the method according to claim 43 or claim 44 wherein, upon identification of a deficiency in one or more beneficial heavy metal(s) in the subject, corresponding heavy metal supplement is/are administered to the subject.

46) The composition for use or the method according to any one of claims 41 to 45, wherein the level of heavy metal(s) in the subject are measured using a spectrometer.

47) The composition for use or the method according to claim 46, wherein the spectrometer is a miniature spectrometer.

48) A method for producing the composition according to any one of claims 1 to 29, the method comprising:
a) mixing the chelating agent with the extended surface biologically inert substrate; and
b) agitating the mixture.

49) The method according to claim 48 wherein agitating the mixture comprises subjecting the mixture to cavitation.

50) The method according to claim 48 or claim 49, wherein agitating the mixture comprises subjecting the mixture to sonication.

51) The method according to claim 50, wherein the sonication is ultrasonication.

52) The method according to claim 51, wherein the ultrasonication is Barbell Horn ultrasonication.

53) A method for producing the composition according to any one of claims 1 to 29, the method comprising synthesising the extended surface biologically inert substrate in the presence of the chelating agent.

54) The method according to claim 53, wherein the extended surface biologically inert substrate is clinoptinolite.

55) The method according to claim 53 or claim 54, wherein the chelating agent is melanin.

56) The method according to claim 55, the method comprising:
a) mixing melanin with an alkaline source, a silica source, and an alumina source; and
b) applying hydrothermal conditions to the mixture.
